(19) United States
(12) Patent Application Publication
(10) Pub. No.: US 2013/0338044 A1
(43) Pub. Date: Dec. 19, 2013

(54) BIOCONJUGATION USING BIFUNCTIONAL LINKERS

(71) Applicant: THE REGENTS OF THE UNIVERSITY OF CALIFORNIA, Oakland, CA (US)

(72) Inventors: Jiayu Liao, Carlsbad, CA (US); Yongfeng Zheng, Riverside, CA (US)

(21) Appl. No.: 13/914,085

(22) Filed: Jun. 10, 2013

Related U.S. Application Data
(63) Continuation of application No. PCT/US2011/064253, filed on Dec. 9, 2011.
(60) Provisional application No. 61/422,123, filed on Dec. 10, 2010.

(51) Int. Cl. G01N 33/552; C07K 17/06; C07F 7/18

(52) U.S. Cl. CPC G01N 33/552; C07F 7/1868; C07K 17/06
USPC 506/32; 534/550; 530/402; 536/26.1; 530/334

(57) ABSTRACT

A bifunctional linker and method of use is provided that has a spacer molecule with a functional group on one end configured to couple to the surface of a substrate and a function group on the other end that is configured couple to a biomolecule and methods of use. The preferred bifunctional linker has a poly(ethylene glycol) spacer ranging from 3 to 20 ethylene glycol units that has a silane functional group to react with a substrate and an azide functional group that can couple to a biomolecule that includes an alkyne group. The preferred linker can produce an azide-derivatized glass surface in one step and the azide functional group of the spacer can in sequence conjugate with a biomolecule using click chemistry, which can be conducted at low temperature and in aqueous solution.

### CROSS-REFERENCE TO RELATED APPLICATIONS

This application is a 35 U.S.C. §111(a) continuation of PCT international application number PCT/US2011/064253 filed on Dec. 9, 2011, incorporated herein by reference in its entirety, which is a nonprovisional of U.S. provisional patent application Ser. No. 61/422,123 filed on Dec. 10, 2010, incorporated herein by reference in its entirety. Priority is claimed to each of the foregoing applications.

The above-referenced PCT international application was published as PCT International Publication No. WO 2012/079030 on Jun. 14, 2012 and republished on Sep. 7, 2012, and is incorporated herein by reference in its entirety.

### STATEMENT REGARDING FEDERALLY SPONSORED RESEARCH OR DEVELOPMENT

This invention was made with Government support under Grant No. A1076504, awarded by the National Institute of Health (NIH). The Government has certain rights in this invention.

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to surface functionalized substrates and microarray fabrication methods, and more particularly to methods for fabricating high density microarrays utilizing bifunctional linkers for click chemistry or other chemical conjugations for use in peptide, protein or other biomolecular microarrays in a variety of applications.

2. Description of Related Art

Biosensors with solid surfaces, such as microarrays, that enable biological assays, diagnostic tests, and high-throughput screening that need only small amounts of analytes have emerged as a prominent and revolutionary technology. Peptide or protein microarrays are highly desirable for use in biomedical research, especially with molecular diagnostics and drug screening applications.

For example, proteases are pervasive and essential for cellular function through their hydrolysis of specific substrates, peptides and proteins. Numerous physiological processes, such as cell growth and differentiation, cell-cell communication, and cell death, are dependent on the actions of proteases. Furthermore, proteases are involved in diverse diseases, such as cardiovascular disease, cancer, AIDS, and neurodegenerative diseases.

Peptide microarrays are excellent tools in the study of proteolytic processing. They have been used as reliable and efficient methods for the rapid analysis of peptide-based assays. Peptides conjugated to microarrays allow literally thousands of peptides to be evaluated in a single assay with minimal quantities of samples and reagents. The highly scalable nature of these assays is very valuable in biological studies and especially in pharmaceutical screening.

A key step in the fabrication of peptide or protein microarrays is the immobilization of biomolecules to glass surfaces (conjugation). High immobilization efficiency is crucial to the use of surface biosensors as quantitative tools for bioassays, as is knowledge of the surface density.

Generally, two methods have been used to prepare peptide microarrays. The first is in situ synthesis where different peptides are synthesized by coupling amino acids onto a solid surface step by step. However, the low efficiency of in situ peptide synthesis limits its use in large scale fabrications. In the second method, peptides are pre-synthesized and thereafter are spotted at different coordinates on the solid surface. However, the most commonly used chemistries for peptide or protein immobilization, such as amide-bond formation and reductive amination, involve random covalent-bond formation with multiple reactive amino acids in peptides or proteins, which inevitably has a substantial negative effect on peptide activity. Therefore, it is very hard to achieve the reproducibility and sensitivity necessary for sensitive, reliable activity-based assays.

In addition, conjugation efficiency is another major concern when peptides are conjugated on to a glass surface. Thus, given the presence of multiple reactive amino acids in peptides or proteins under physiological conditions, and the current limitations to immobilization efficiency, there is a considerable need for highly selective and efficient orthogonal reactions.

Accordingly there is a need for a method for producing high density microarrays that has high peptide immobilization efficiency and that is sensitive, reproducible and reliable. A need also exists for a method which renders a low cost, scalable process for producing microarrays. The present invention satisfies these needs, as well as others, and is generally an improvement over the art.

### BRIEF SUMMARY OF THE INVENTION

The present invention provides a one-step modified glass substrate surface that can then be used for chemoselective covalent coupling of an alkyne terminated peptide or other biomolecule by click chemistry and its applications in creating peptide microarrays etc. By way of example, and not of limitation, the subject invention includes the use of a bifunctional linker with a spacer molecule that has a functional group to react with a substrate on one end and a functional group that can couple to a biomolecule on the other end. The preferred linker reagent can modify glass surfaces in one step to produce functionalized glass surface with anchored spacers with an azide group. The azide group functionalized glass surface is ready to immobilize a peptide, via a click reaction approach, thereby producing chemically and biologically orthogonal patterns or arrays.

The synthesis of one embodiment of a bifunctionalized triethoxysilane derivative is illustrated in FIG. 3. Generally, the bifunctionalized reagent in that illustration is synthesized with tetraethylene glycol as a starting material. The allyl group is introduced by a desymmetrization reaction with allyl bromide in a basic condition. The free hydroxyl group is converted into the azide group by treating with tetrabromide carbon and sodium azide, sequentially. Hydrosilylation is finally carried out using triethoxysilane in the presence of Karstedt catalyst to obtain the desired silane with the azide group intact. Hydrosilylation of the double bond is performed in the last step to avoid unnecessary hydrolysis and condensation reactions of the labile triethoxysilane functionality.

The preferred bifunctional reagent linker has a spacer with a silane functionality on one end and an azide functionality on the other end. One preferred biocompatible spacer is poly(ethylene glycol) (PEG) because it is known to be generally inert and provides a hydrophilic spacer between the substrate surface and the active molecule of interest. The length of the spacer chain of the linker can vary depending on the size of the peptide or other molecule that is attached at the distal end of the linker. However, a length of three to twenty ethylene glycol units is preferred.

Click reactions are preferred to conjugate biomolecules because the click reaction can rapidly achieve high yields, and more importantly, it is completely compatible with aqueous conditions. To control the orientation of the attachment, the two terminal groups on the spacer are unique and not cross reactive and can therefore react with a glass surface and biomolecules sequentially. Click chemistry has been successfully applied to the conjugation of a peptide containing an alkyne group onto the azide derivatized glass surface which was prepared using the bifunctional molecule in one step. A very high density of peptides (1.3×10¹⁴ peptides/cm²) on the glass surface was obtained using this strategy.

In addition, it is evident that for various array-based applications involving peptides, much cleaner and stronger results may be obtained when the peptides are extended further away from the surface of the array. For example, proteases have high requirements for displaying bioactivity, and they are detected not only by their binding activities but also by their enzymatic activities. The use of the linker will permit better accessibility of the immobilized biomolecules to reactive enzymes etc.

Accessibility and bioactivity of peptides immobilized on the glass surface was demonstrated by selective cleavage of peptides using the protease, trypsin. In addition, the advantages of site-specific immobilization of peptides containing an alkyne group, which can be easily incorporated into a peptide by solid peptide synthesizer, were illustrated.

When peptides were conjugated onto the surface in an orthogonal manner using the present methods, the active sites of the peptide were preserved. Therefore, the bioactivity of the peptide was substantially higher than that observed by random amide-bond formation approaches known in the art. In addition, due to the high efficiency of the click reaction, the peptide can be immobilized on the glass surface in a uniform density, which was proportional to the concentrations of peptide in solution. Given the high efficiency and very biocompatibility of this site-specific conjugation approach, the procedure is suitable for the fabrication of peptide and protein microarrays with well-developed DNA array facilities.

The invention may be utilized in several ways, including, but not limited to the following embodiments:

A first embodiment comprises a surface modifying agent for the attachment of a biomolecule to a substrate, comprising a spacer with a substrate surface conjugating functional group attached to one end of the spacer configured to couple with a substrate surface and a biomolecule conjugating functional group attached to the other end of the spacer that is configured to couple to a biomolecule.

A second embodiment comprises a glass surface modifying agent utilized for peptide attachment that includes: a linker or spacer having first and second ends; an azide function group attached to the first end of the spacer; and an alkoxysilane functional group attached to the second end of the spacer.

A third embodiment comprises a glass surface modifying agent utilized for alkyne-derivatized peptide attachment that includes a polyethyleneoxide spacer having first and second ends; an azide functional group attached to the first end of the spacer; and a trialkoxysilane functional group attached to the second end of the spacer.

A fourth embodiment comprises a glass surface modifying agent according to the second embodiment, wherein the trialkoxysilane function group is a triethoxysilane functional group.

A fifth embodiment comprises a method of attaching a peptide to a glass surface that includes the steps: silanizing a glass surface with a modifying agent; and conjugating an alkyne-derivatized peptide with the silanized glass surface. The modifying agent comprises: a spacer having first and second ends; an azide functional group attached to the first end of the spacer; and an alkoxysilane functional group attached to the second end of the spacer.

Additionally, it must be appreciated that the subject system of conjugation may be utilized in many varied applications and with many varied molecules in equivalent fashion to the applications described below that are specifically for peptides. Carbohydrates and carbohydrate derivatives, lipids and lipid derivatives, nucleotides, and nucleotide derivatives may be conjugated to supports, along with acceptable organic molecules of varied other characterizations. Various supports may be utilized, including, but not limited to glass surfaces of any structural configuration, beads, cylinders, microarray elements, high throughput screening components, microfluidic components, medical devices, general manufactures, and products. Various biotransformation applications may also find the subject derivatives appropriate.

Furthermore, the subject conjugation method using a bifunctional linker can be used to conjugate alkyne-containing molecules to various surfaces, such as glass surface, a gold surface, a titanium surface, a silica gel surface, polymer beads, a silicone surface, agarose beads, and metallic oxide-based nanoparticles.

Further aspects and embodiments of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a schematic diagram of the formation of a microarray construct with a bifunctional linker with a silane functionality that binds to a substrate and an azide functionality that binds to a biomolecule according to one embodiment of the invention.

FIG. 2 is a schematic diagram of the formation of a microarray construct with a bifunctional linker with a silane functionality that binds to a substrate and an azide functionality that binds to a marked peptide that is specific for a peptide according to one embodiment of the invention.

FIG. 3 is a schematic diagram of a synthesis scheme for one embodiment of a bifunctional linker reagent with a poly(ethylene glycol) spacer and a trialoxysilane and azide functional groups according to the invention.

### DETAILED DESCRIPTION OF THE INVENTION

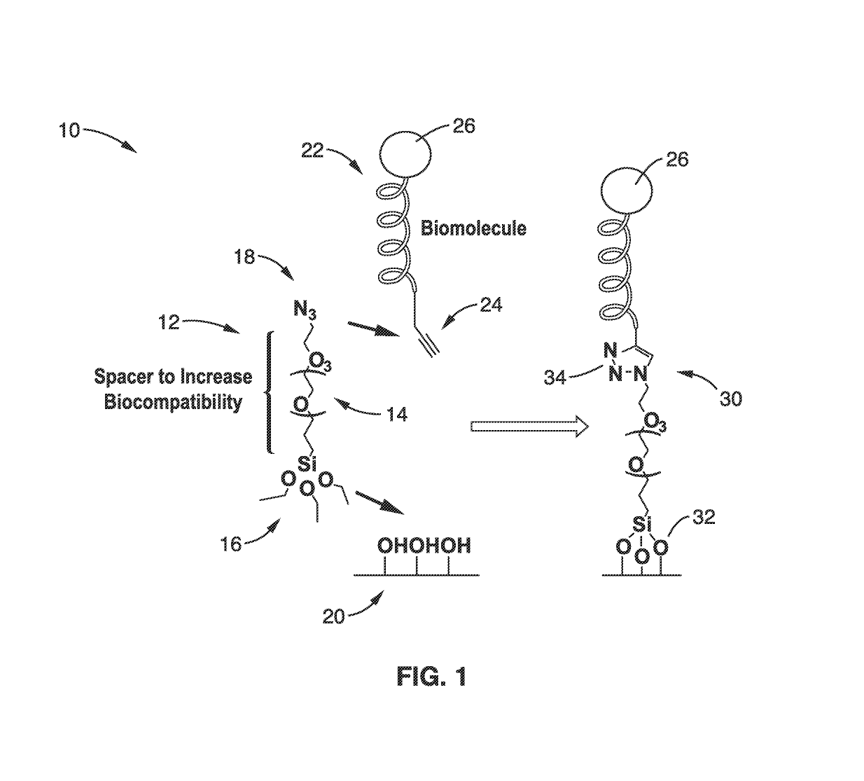

Referring more specifically to the drawings, for illustrative purposes the present invention is embodied in the apparatus and methods generally illustrated in FIG. 1 through FIG. 3. It will be appreciated that the apparatus embodiments may vary as to configuration and as to the details of the parts, and that the methods may vary as to the specific steps and sequence, without departing from the basic concepts as disclosed herein.

The present invention provides a simple method for producing a microarray or other functionalized surface. Peptide microarrays, for example, are an excellent tool for the study of proteolytic processing and are used as an illustration of the reagent constructs and methods.

The surface functionalization of a substrate such as a glass surface begins with the production of a bifunctional coupling molecule that has a spacer with at least one functional group to react with a substrate at one end and at least one functional group that can couple to a biomolecule on the other end. The preferred bifunctional linker has a spacer with a silane termini and an azide termini and this molecule is preferably used to prepare an azide-derivatized glass surface in one step. The resulting glass surface is available for fabricating a peptide microarray by the conjugation of the azide terminus of the spacer with an alkyne containing peptide or other molecule using click chemistry, which can be conducted at low temperature and in aqueous solution.

The high density of peptides or other active molecules coupled to the substrate surface is achieved by the concise conjugation reaction of the bifunctional linker molecule that is very efficient and produces an even array of oriented active biomolecule elements. For example, due to the biologically orthogonal manner of conjugation, peptides conjugated by the site-specific immobilization methods are shown to be far more accessible to protease than peptides conjugated by random amide conjugation. High immobilization efficiency and predictable surface density are also crucial to the use of surface biosensors as quantitative tools for bioassays.

In addition, it is evident that for various array-based applications involving peptides, much cleaner and stronger results may be obtained when the peptides are extended further away from the surface of the array. For example, proteases have sensitive requirements for displaying bioactivity, and they are detected not only by their binding activities but also by their enzymatic activities. The use of a bifunctional linker 12 will permit better accessibility of the immobilized biomolecules to reactive enzymes etc.

Turning now to FIG. 1, the production of a microarray or other functionalized substrate surface 10 is achieved initially with the production of a bifunctional linker reagent 12. Generally, the bifunctional linker reagent 12 is a surface modifying agent that facilitates the attachment of a biomolecule to a substrate. The linker reagent comprises a spacer with first and second ends with at least one functional group present at both ends. The functional group attached to one end of the spacer couples with a substrate surface and the functional group attached to the other end of the spacer conjugates to a biomolecule. The preferred surface conjugating functional group of the spacer is a silane such as an alkoxysilane, a trialkoxysilane, or triethyloxysilane. However, the surface conjugating functional group can also be dihydrogen phosphate, a thiol group or an alkyne group. The biomolecule conjugating functional group of the spacer is preferably an azide. However, an alkene, a ketone, an aldehyde, an ester, a carbamate or a phosphane can also be used as a biomolecule conjugating functional group of the spacer 14.

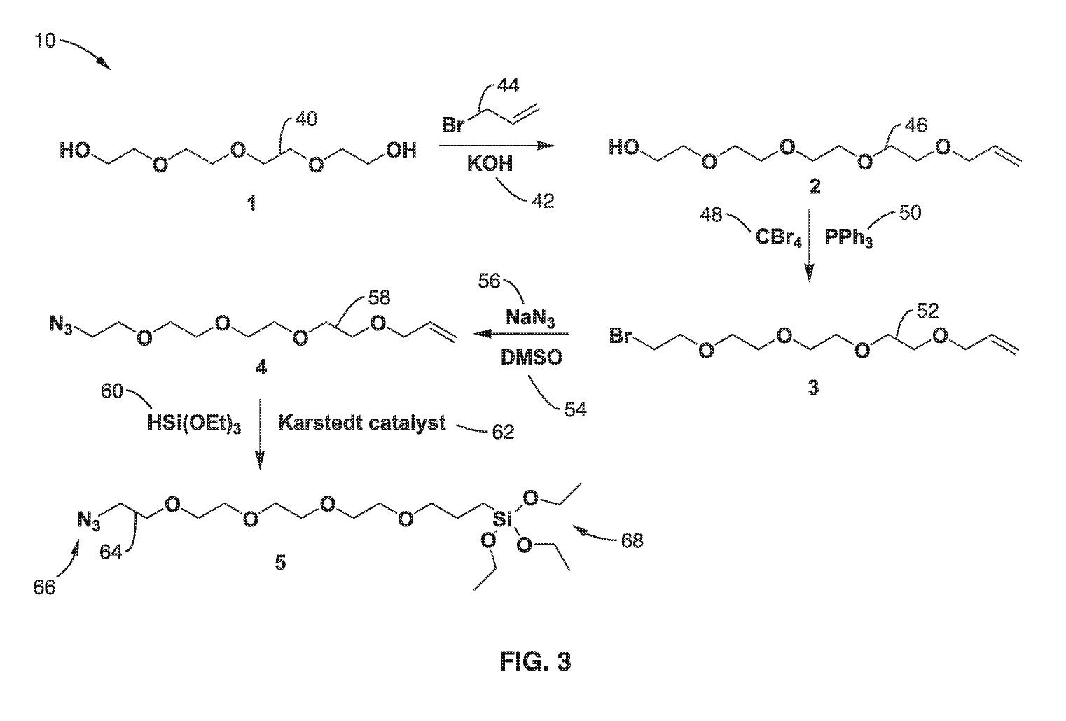

In the embodiment shown in FIG. 1, the bifunctional linker reagent 12 has a silane functionality 16 and an azide functionality 18 on each end of a spacer or linker 14 molecule to illustrate the invention. The synthesis of one embodiment of a bifunctionalized triethoxysilane derivative a bifunctional linker reagent 12 is illustrated in FIG. 3.

The spacer 14 of the linker reagent 12 is preferably a biocompatible spacer such as poly(ethylene glycol) (PEG) because it is known to be generally inert and provides a hydrophilic spacer between the substrate surface and the molecule of interest. PEG is flexible and permits conjugation without steric interference. The linker structure 12 also brings the peptide or other biomolecule away from the substrate surface 20 without interfering with the biological activity of the peptide 22 or other molecule that is attached to the linker.

The length of the spacer chain 14 of linker 12 can vary depending on the size of the peptide or other molecule 22 that is to be attached at the distal end of the spacer 14. The preferred length of the PEG oligomer spacer 14 ranges from between 3 to 20 ethylene glycol units with a range of 3 to 5 units particularly preferred. Although a modified PEG linker is preferred, it will be understood that other types of linkers can be used. For example, other biocompatible linkers, such as peptide, peptide mimics, nucleotide mimics and biopolymers.

To control the orientation of the attachment to both the substrate 20 and the biomolecule 22, the two terminal groups on the spacer 14 of linker 12 are unique and are not cross reactive and therefore the reactions with a glass substrate surface 20 and biomolecules 22 can take place sequentially.

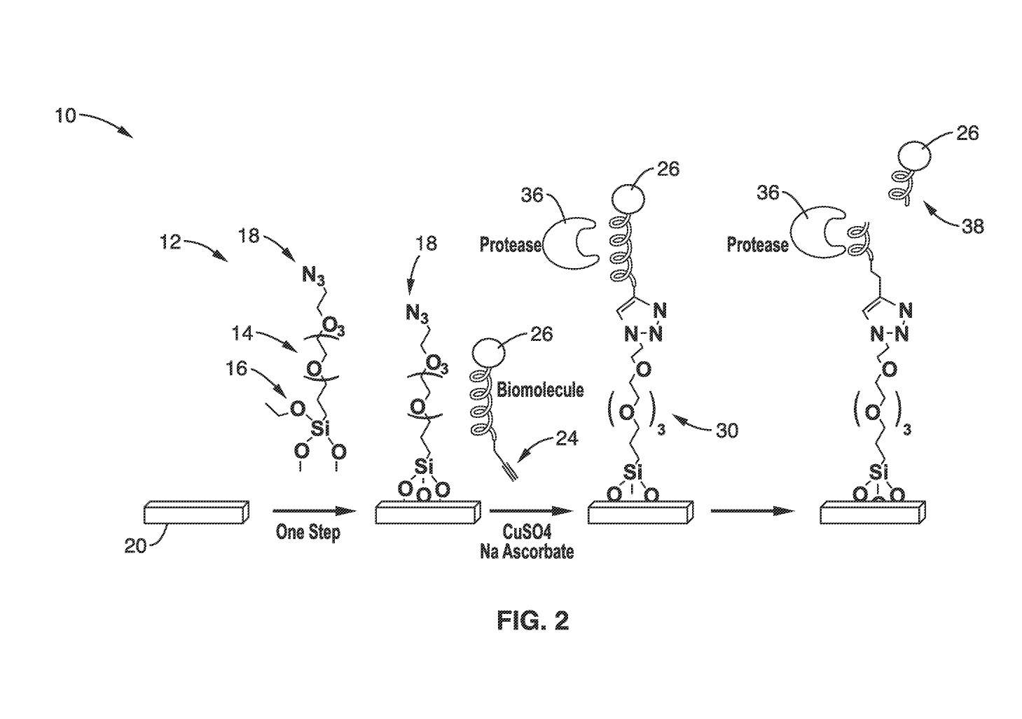

A functional silane group 16 is formed at one end of spacer 14 of linker molecule 12. In the embodiment shown in FIG. 1 and FIG. 2, a triethoxysilane derivative is used as the surface functional group 16. However, an alkoxy silane functional group or a trialkoxysilane functional group is preferably used. However, a dihydrogen phosphate, a thiol group or an alkyne group can also be used as the surface functional group 16.

The silane functionality 16 couples the linker reagent 12 to the surface of the substrate 20. Substrate 20 is preferably made of glass with a smooth surface. However, substrate 20 can also be a silica surface, a silica gel surface, silicone surface or a metal surface, such as a gold or titanium surface. The substrate 20 may also take different structural forms in addition to a planar glass surface such as, polymer beads, agarose beads, or metallic oxide-based nanoparticles, cylinders, microarray elements, microfluidic components, or screening components for example.

At the other end of the spacer 14 is preferably an azide group 18 that is capable of conjugating with an alkyne functional group 24 on a biomolecule 22 or other desired molecule. In the scheme shown in FIG. 1 and FIG. 2, a triazole is formed from the 1.3 dipolar cycloaddition of azide and alkyne to covalently bond the peptide 22 to the linker 12 and the substrate 20. The biomolecule conjugating functional group of the spacer is preferably an azide. However, an alkene, a ketone, an aldehyde, an ester, a carbamate or a phosphane can also be used as a biomolecule conjugating functional group of the spacer 14.

Although a peptide is described as the biomolecule 22 that is attached to the linker reagent 12 as an illustration, it will be understood that any type of molecule can be coupled to the azide functionality 18, directly or indirectly. For example, the biomolecule 22 can be a carbohydrate or carbohydrate derivative; a lipid or lipid derivative, a nucleic acid, a nucleic acid protein complex and other organic or inorganic molecules or compounds.

As seen in FIG. 1, the fundamental element of the surface functionalization or microarray is the construct 30 comprising a spacer 14 with a silane functionality 16 anchored the substrate 20 at one end with bond 32 and to a biomolecule 22 attached to the linker 12 with bond 34.

In one embodiment, an optional marker 26, such as a fluorescent label can be attached to the biomolecule 22 to allow the visualization of the attached elements of the array. The use of NBD, 7-nitrobenz-2-oxa-1,3-diazol-4-yl, as a fluorescent marker 26 for immobilized peptides is particularly preferred. Because of the small size of NBD, this fluorophore can be included in an amino acid that is incorporated into any position of a peptide via a conventional automated solid-phase peptide synthesis. Other fluorescent groups can also be used as marker for monitoring the conjugation process and results as well as a measurement for bioactivity.

In addition, a linear orientation of the biomolecule with respect to the length of spacer 14 can be created by the placement of an alkyne 24 element on the terminal of the peptide or other biomolecule 22. The controllable orientation and placement of the biomolecule 22 in three dimensions make the construct 30 particularly suitable for the production of microarrays as well as larger scale arrays.

Referring now to FIG. 2, one embodiment of a method 10 for producing a protease activity array is schematically shown. The first step is the formation of a bifunctional linker reagent 12 with a spacer 14 that includes an alkoxysilane functionality 16 at one end and an azide functionality 18 at the other in this illustration. A triethoxysilane linker reagent 12 is illustrated in FIG. 2.

Optionally, the second step is to hydrate the surface of the substrate 20. With glass substrates 20, hydration is preferably performed with a 5:1 solution of H₂SO₄/H₂O₂.

The third step is to expose the substrate 20 to the bifunctional linker reagent 12 to bond the linker 12 with the substrate 20. In this configuration, the bifunctional reagent 12 can modify glass substrate surfaces 20 in one step and produce a functionalized glass surface densely populated with bound linkers with at least one open azide group 18 at the distal end.

The fourth step is to provide a biomolecule 22 that includes an alkyne group 24. The biomolecule 22 shown in FIG. 2 also has a fluorescent label or other type of marker 26. The biomolecule 22 used in the illustration has a sequence that can be cleaved by a protease 36.

The fifth step is to couple the selected biomolecule 22 to the anchored linkers through the azide functionality 18 and the alkyne group 24 of the biomolecule 22. Following the silanization step, the immobilization reaction of the peptide biomolecule 22 is preferably performed by azide-alkyne cycloaddition. This step completes the basic array component.

In the scheme shown in FIG. 2, the presence of the construct 30 on the substrate 20 can be seen by observing the fluorescence of label 26. The accessibility and bioactivity of peptide 22 immobilized on the glass surface can be demonstrated by selective cleavage of peptide 22 using a protease 36 such as trypsin. Exposure of the peptide to a protease 36 results in proteolysis of the peptide. The cleaved portion 38 of the peptide has the label 26 so that fluorescence is diminished or eliminated by the action of the protease and removal of the label 26. This approach demonstrated that the active sites of the peptide are preserved when the peptide 22 was conjugated onto the surface in an orthogonal manner using the present methods. Therefore, the bioactivity of the peptide is consistent and substantially higher than that observed by random amide-bond formation approaches known in the art.

In addition, due to the high efficiency of the click reaction, the peptide can be immobilized on the glass surface in a uniform density, which is proportional to the concentrations of peptide in solution. Given the high efficiency and biocompatibility of this site-specific conjugation approach, the procedure is suitable for the fabrication of peptide, protein and other biomicroarrays and with well-developed DNA array facilities.

The invention may be better understood with reference to the accompanying examples, which are intended for purposes of illustration only and should not be construed in any sense as limiting the scope of the present invention as defined in the claims appended hereto.

### Example 1

In order to demonstrate the invention, a synthesis scheme for one embodiment of a bifunctional reagent was produced and evaluated. The synthesis scheme of a bifunctionalized triethoxysilane derivative embodiment is illustrated in FIG. 3. Generally, the bifunctionalized reagent (Compound 5) of FIG. 3 is synthesized from the starting material tetraethylene glycol 40. The allyl group is introduced by a desymmetrization reaction with allyl bromide 44 in basic conditions. The free hydroxyl group was converted into the azide group by treating with carbon tetrabromide 48 and sodium azide 56, sequentially. Hydrosilylation is finally carried out using triethoxysilane 60 in the presence of a Karstedt catalyst 62 to obtain a silane (Compound 5) with the azide group intact. Hydrosilylation of the double bond is performed in the last step to avoid unnecessary hydrolysis and condensation reactions of the labile triethoxysilane functionality. The silane (Compound 5) and intermediates that were prepared here were characterized by ¹H and ¹³C NMR and mass spectrometry.

Referring specifically to FIG. 3, it can be seen that there are three intermediate compounds produced in the scheme that results in novel dual functional silane 64 with the tetraethylene glycol spacer that can be used for preparing a peptide microarray. In this illustration, (1.94 grams, 10 mmol) tetraethylene glycol 40 (Compound 1) was placed in a flame-dried flask. Then KOH 42 (0.561 grams, 10 mmol) was added slowly to the flask. The resulting mixture was stirred quickly until the KOH 42 was dissolved. While keeping the flask in a water bath at room temperature, 1.2 grams of 10 mmol allyl bromide 44 was added drop wise to the mixture. The resulting mixture was stirred at room temperature for 2 hours, and then at 60° C. overnight. The reaction mixture was dissolved in water and extracted with dichloromethane. The organic phase was then dried over anhydrous Na₂SO₄. After evaporation of solvent, the residue was purified by flash chromatography on silica gel (EtOAc, then eluted with EtOAc:MeOH=10:1) to produce the first intermediate 46 (Compound 2) (1.02 g, 4.35 mmol, 43.5%) as colorless oil. ¹H NMR (CDCl₃, 25° C.) δ d 2.51 (bs, ¹H), 3.60-3.80 (m, 16H), 4.01 (m, 2H), 5.18 (dd, J=10.0 Hz, J=1.6 Hz, 1H), 5.26 (dd, J=17.2 Hz, J=1.6 Hz, 1H), 5.91 (m, 1H). ¹³C NMR (CDCl₃, 25° C.) δ 61.72, 69.37, 70.25, 70.55, 70.61, 72.25, 72.67, 117.25, 134.61.

To the flask with Compound 2 (0.40 g, 1.71 mmol) in THF (6 mL), 0.83 grams of 2.5 mmol carbon tetrabromide 48 and 0.65 grams of 2.5 mmol triphenylphosphine 50 were added successively to the flask in an ice water bath. The resulting mixture was stirred at room temperature overnight. After the solvent was removed, the residue was partitioned in water and dichloromethane. The organic phase was washed by water and dried over anhydrous Na₂SO₄. After evaporation of solvent, the residue was purified by flash chromatography on silica gel (EtOAc:Hexanes=3:2) to get the second intermediate 52 (Compound 3) (0.43 g, 1.45 mmol, 84.8%) in the form of a pale yellow oil. ¹H NMR (400 MHz, CDCl₃, 25° C.) δ 3.46 (t, J=6.4 Hz, 2H), 3.58-3.62 (m, 2H), 3.63-3.70 (m, 12H), 3.80 (t, J=6.4 Hz, 2H), 4.02 (m, 2H), 5.17 (dd, J=10.8 Hz, J=2.0 Hz, 1H), 5.26 (dd, J=17.2 Hz, J=2.0 Hz, 1H), 5.90 (m, 1H). ¹³C NMR (50 MHz, CDCl₃, 25° C.) δ 30.27, 69.40, 70.49, 70.61, 71.16, 72.19, 117.04, 134.73.

To Compound 3 (2.15 g, 7.23 mmol) of FIG. 3 held in DMSO 54 (10 mL), 0.8 grams of 12.3 mmol sodium azide 56 was added. The resulting mixture was stirred at room temperature overnight. Then the mixture was partitioned between water and dichloromethane. The organic phase was washed with water and dried over anhydrous Na₂SO₄. After evaporation of solvent, the residue was purified by flash chromatography on silica gel (EtOAc:Hexanes=3:2) to get a third intermediate 58 (1.7 g, 6.55 mmol, 90.6%) as colorless oil (Compound 4). ¹H NMR (400 MHz, CDCl₃, 25° C.) δ 3.37 (t, J=5.0 Hz, 2H), 3.56-3.62 (m, 2H), 3.63-3.70 (m, 12H), 4.01 (d, J=6.4 Hz, 2H), 5.17 (dd, J=9.6 Hz, J=1.6 Hz, 1H), 5.26 (dd, J=17.2 Hz, J=1.6 Hz, 1H), 5.90 (m, 1H). ¹³C NMR (50 MHz, CDCl₃, 25° C.) δ 50.66, 69.38, 69.99, 70.61, 72.19, 117.05, 134.74.

To a 1 mL solution of Compound 4, 0.13 grams of 0.50 mmol in triethoxysilane 60, was added along with 30 μL of a Karstedt catalyst 62 (2.1-2.4% platinum (0)-1,3-divinyl-1,1,3,3-tetramethyldisiloxane complex dissolved in xylene). After the resulting solution was stirred at 60° C. overnight, the triethoxysilane 60 was removed under vacuum. Then 0.5 mL of anhydrous toluene was added, and removed again. The procedure was repeated three times to eliminate traces of triethosilane. The residue 64 was used directly in the silanization step without any further purification. For purpose of analysis, a little of residue was dissolved in CDCl₃. ¹H NMR (400 MHz, CDCl₃, 25° C.) δ 0.62 (m, 2H), 1.21 (t, J=6.8 Hz, 9H), 1.69 (m, 2H), 3.38 (t, J=5.0 Hz, 2H), 3.43 (t, J=6.8 Hz, 2H), 3.58 (m, 2H), 3.60-3.70 (m, 12H), 3.81 (q, J=6.8 Hz, 2H). ¹³C NMR (50 MHz, CDCl₃, 25° C.) δ 6.42, 18.27, 22.88, 50.69, 58.34, 70.02, 70.61, 73.66. MS (ESI): M_{calcd}=423.2401. found (m/z): 441.2745 (M+NH₄⁺).

Hydrosilylation of the double bond is preferably performed in the last step to avoid unnecessary hydrolysis and condensation reactions of the labile triethoxysilane functionality. The final silane material 64 (Compound 5) that was prepared was characterized by ¹H and ¹³C NMR and mass spectrometry. The triethyloxysilane group 68 and azide group 66 of Compound 5 were both confirmed.

### Example 2

In order to demonstrate the single step functionalization of a substrate and peptide immobilization illustrated in FIG. 1, the Compound 5 linker 64 was prepared using the scheme of FIG. 3 and used on a glass slide substrate.

Step 1. Hydration of the glass slide surfaces: The glass slides were dipped in piranha solution (5:1 H₂SO₄/H₂O₂) for overnight and rinsed with deionized water. The glass slides were then dried under Argon gas.

Step 2. Silanization with Compound 5: The solution of compound 5 in toluene was filtered by PTFE filter (Fisherbrand, 0.45 μm). The glass slides were dipped in a 10 mM solution of compound 5 in toluene for overnight storage at room temperature. The slides were then washed with toluene, ethanol, THF, and deionized water, then in the reversed order.

The bifunctional reagent 64 (Compound 5) was then conjugated onto the glass surfaces in toluene solution. The silanization step was followed by a curing at 80° C. for 3 hours to stabilize the silane layer. Contact angle θ was measured to be 41.3±1.8° for the modified glass slides using water. On the other hand, the θ value of unmodified slide was found to be less than 10° using water. This value was similar to contact angle values for surfaces after modification with PEG segment. The result showed that the reagent was well conjugated on the surface. This coating was found to be stable at least six months when storing at 4° C.

Step 3. Conjugation of peptide on the substrate surface (as illustrated in FIG. 1): Following the silanization step, the immobilization reaction was performed by azide-alkyne cycloaddition. To demonstrate the conjugation of peptide with the linker, a peptide with an alkyne group on the N-terminus and an unnatural amino acid containing 7-nitrobenz-2-oxa-1,3-diazole (NBD) unit on the other end of the peptide was designed. The NBD was a small fluorophore and had high quantum yield and longer wavelength emission maxima which was compatible with most scanner machines. In addition, the peptide was synthesized on a regular peptide synthesizer without any post-modification.

The selected biomolecule was a peptide that was a trypsin sensitive peptide including a porcine homologue to human Fc gamma RIIIA alpha and NBD is the NEMO-binding domain. The peptide was spotted on the glass slide (azide derivatized glass slide with —N₃) and covered with another plain glass slide with parafilm as a support. Conjugation with 1 mM and 0.5 mM peptide was then conducted. For the conjugation solution of 1 mM peptide, the solutions consisted of PBS buffer (pH=8.0) containing 1.0 mM peptide, CuSO₄ (20 μM, 0.02 equal of peptide), and Na Ascorbate (400 μM, 0.2 equal of peptide). After a slide was incubated at room temperature for 0.5 hours, each slide was washed with water, detergent solution (either 0.1% Tween 80 and or 0.5% Triton X-100), deionized water, and then ethanol. The slides were finally dried with a stream of Argon gas.

The presence of an active azide group on the glass slide after being modified by the bifunctional reagent (Compound 5) was demonstrated. The immobilization of a fluorescence labeled-peptide onto an azide derivatized glass slide was verified with representative fluorescence images.

The availability for cleavage of the anchored peptides by protease was then evaluated. The peptide array was covered with cover slip. Hydrolysis reactions were initiated by pipetting either trypsin solution or control solution between the glass slides. The trypsin solution consisted of 0.5 mg/mL trypsin in Tris Buffer (Tris-HCl 20 mM, NaCl 50 mM, pH 7.5). Tris Buffer or BSA (0.5 mg/mL) in Tris Buffer was used as control solution. The reactions were terminated by washing the slides with deionized water after maintaining the reactions at room temperature for 3 h. Fluorescence intensity ratio (FIR) was calculated by ratio of fluorescence intensity obtained after trypsin digestion and that obtained before trypsin digestion.

Peptides were spotted onto the glass surface and incubated for half an hour and were extensively washed and only the alkyne-containing fluorogenic peptides were able to be immobilized on the modified glass surface under the conditions of click chemistry. In contrast, the control peptide containing amine group was not able to be conjugated on the glass surface under the same conditions. This demonstrated that azide groups on glass slides after modification by the bifunctional reagent were available for cycloaddition only with the alkyne group. No fluorescent signal from fluorogenic peptide was observed when copper catalyst or sodium ascorbate was absent. These results further demonstrated that the conjugation was specific for the azide-alkyne cycloaddition. The conjugation can be finished in 30 min at room temperature.

The quantitative range of peptide that can be immobilized on glass surface by the azide-alkyne cycloaddition method was then investigated. The maximum density of peptides immobilized on the surface was calculated to be about 1.3×10¹⁴ peptides/cm². This density was equivalent to the maximum peptide molecules which occupied the surface area in a vertical orientation. The density of peptides that were obtained was substantially higher than that prepared by other conjugation methods. The value was comparable with carbohydrate and DNA arrays.

Peptide concentrations of 100 to 1,000 μM in PBS containing CuSO₄ and sodium ascorbate resulted in a very good linear relationship of peptide concentration and fluorescence signal on the glass surface. The linear relationship of peptide concentration to the fluorescence signal of immobilized peptide demonstrated the robustness of the immobilization reaction. The broad range of peptide substrate could be used not only to measure enzyme activity quantitively, but might also potentially be used to measure its kinetics quantitatively.

### Example 3

In order to monitor bioconjugation efficiencies and bioactivities with the NBD-containing peptide, a bifunctional peptide with NBD at the C-terminus and an alkyne group at the N-terminus was produced. The alkyne group was conjugated onto a glass surface bearing an azide group using click chemistry and the surface-bound peptide was detected by a microarray scanner.

Furthermore, a fluorescent peptide substrate for trypsin, which can be cleaved at the carboxyl side of the amino acids lysine and arginine, was also conjugated to the surface by this procedure. Accessibility of the peptide on the surface to enzymatic reactions was demonstrated by its ready cleavage by trypsin. No cleavage was detected when BSA was used as a control. Enzyme activity was easily observed by a decrease of fluorescence on the surface image. Importantly, no blocking step was needed after peptide conjugation or before protease digestion. It is likely that the azide group on the glass surface was inert under most of the reaction conditions.

The optimum reaction conditions of for bioconjugation were also evaluated. An azide-derivatized surface was prepared by silanization with the synthetic reagent that was developed that includes a polyethylene glycol (PEG) linker and a terminal azide group. To determine the optimal immobilization time, bifunctional peptides with an alkyne group and a Dap(NBD) at opposite ends were printed on a glass substrate. The Huisgen click cycloaddition reaction was performed under humid conditions for 0.5-6.0 hours. The conjugation solution was phosphate-buffered saline (PBS, pH 8.0) including 1.0 mM peptide, CuSO4 (0.10 mM), sodium ascorbate (2.0 mM), and glycerol (10% v/v, to impede evaporation). Slides incubated for 30 min showed high-intensity fluorescence.

Some studies have suggested conjugation efficiency can be improved by prolonging the time of incubation. However, it was observed that 30 minutes was optimal for peptide conjugation. In fact, the amount of peptide conjugated decreased slightly when longer incubations were used. The copper and sodium ascorbate might also adversely affect the NBD peptides.

The effects of pH and the concentration of copper and sodium ascorbate on the immobilization were also evaluated. The pH was varied from 6.0 to 9.0, while keeping the incubation time fixed at 30 min. It was found that pHs of 7 and 8 were best for efficient immobilization. This pH range is also optimal for preserving the biological activities of most peptides. The effect of copper concentrations of 0.02-0.5 mM with 20 times sodium ascorbate were also evaluated. The fluorescent intensity representing the immobilized NBD-containing peptide increases as the copper concentration increases. The highest fluorescence intensity of conjugated peptides is observed at 0.5 mM copper.

The protease substrate specificity and the survival of biological activity of peptides anchored with the reagent were then evaluated. Once the conjugation protocol had been optimized, it was important to insure that the biological activities of the conjugated molecules were maintained for bioassays. To demonstrate that fluorogenic peptides conjugated on the glass surface were accessible for enzymatic reactions, peptide arrays were first subjected to proteolysis by trypsin which can cleave peptide at carboxyl side of amino acid Lysine or Arginine. In other conjugation methods, a layer of BSA was often required to increase the accessibility of protease to its slide-bound peptide substrates. It was expected that peptide arrays conjugated by the present approach could provide accessibility for enzymatic reactions without BSA present due to the PEG spacer between peptide and surface.

A fluorogenic peptide substrate for trypsin was conjugated to the glass surface using the procedure described above. The accessibility of the peptide array to enzymatic reactions was first demonstrated by cleavage of the peptide by trypsin, whereas the peptide array showed no cleavage when exposed to BSA as a control. It should be noted that no blocking step was needed both after peptide conjugation and before protease digestion. That was probably because the azide group on the glass surface was inert within a majority of organic reaction conditions.

Next, two fluorogenic peptides, for trypsin and sentrin-specific protease 1 (SENP1), respectively, were printed. As expected, the trypsin peptide array showed complete cleavage by trypsin, in contrast to the SENP1 substrate which showed no detectable cleavage, demonstrating that the high fidelity of protease-substrate remained on the glass surface. The peptide immobilized on surface can not only be cleaved but was also cleaved with high specificity. The very efficient cleavage of the slide-bound peptide demonstrated a greater accessibility and sensitivity of the peptide present on the glass surface. The results suggest that fluorogenic peptides can be used for protease substrate specificity testing and also for quantitative digestion determination on glass surfaces.

### Example 4

The efficiency of conjugation is an important factor in making microarrays. To demonstrate the efficiency of the methods to bind biomolecules to solid surfaces, a comparison of the conjugation efficiencies of click chemistry and two traditional methods, amide bond conjugation and hydrazide conjugation, were conducted. Three peptides were prepared. Each peptide was prepared with a functional group appropriate for the conjugation chemistry at its N-terminus. Three different surfaces and conjugation procedures were conducted and compared.

After washing, the three slides were scanned, and the fluorescent intensities of the conjugated peptides were evaluated. The slides were evaluated with a range of different threshold settings of the fluorescent scanner. At the filter threshold setting of zero, fluorescent signals were detected in all three conjugation methods. The fluorescent image of peptides conjugated by the methods of the invention showed a solid spot, while those prepared by the other methods showed only circular shapes. When the filter threshold setting was increased to 280, the fluorescent image of amine-conjugated peptides lost most of its signal, and the keto-conjugated peptides lost signal completely. However, the click chemistry-conjugated peptide still had strong signal.

When the filter threshold setting was increased to 520, only the click chemistry-conjugated peptide still gave strong fluorescent signals. Clearly, the conjugation by click chemistry had the highest fluorescent signals.

### Example 5

In order to monitor bioconjugation efficiencies and bioactivities with the NBD-containing peptide, a bifunctional peptide with NBD at the C-terminus and an alkyne group at the N-terminus was produced.

To demonstrate the utility of the unique physical and chemical properties of 7-nitrobenz-2-oxa-1,3-diazol-4-yl (NBD) as a fluorescent marker for immobilized peptides provided by the methods, several peptide substrates for proteases were prepared and marked with NBD at different locations along the peptide chain. Because of the small size of NBD, it can be included in an amino acid that is incorporated into any position in a peptide with the use of a conventional automated solid-phase peptide synthesizer.

The excitation wavelength of NBD also fits very well with the commonly used blue laser light source. With the use of a fluorescent NBD amino acid, new protease substrates were developed that are attractive because of their excellent chemical stability and long wavelength of excitation (480 nm) of the NBD fluorophore. The fluorescent peptides can be synthesized by Fmoc solid-phase peptide synthesis. Example peptides were efficiently immobilized onto a microarray surfaces using click chemistry, and its proteolysis was monitored by fluorescence imaging. Excellent site specificity was achieved for the protease. Fluorescent peptides are also used to monitor the conjugation efficiency onto a surface using a standard microarray scanner.

The NBD-containing fluorescent peptide solution was printed on about 4000 individual spots on a glass slide with a standard microarray spotter. With a delivered solution volume of 1 nL, the spot size was ca. 200 Lm in diameter, and the distance between spots was 500 Lm. The fluorescent peptide array was ready for evaluation immediately following fabrication. This provides an important improvement over other approaches because the conjugation efficiency for each spot can be monitored by its fluorescence density, and therefore, amount of conjugated peptides/proteins may be easily determined before or after biological reactions.

The peptide array area was covered with cover slip to evaluate cleavage of the peptides by protease. Hydrolysis reactions were initiated by pipetting either trypsin solution or a control solution between the slide and the cover slip. The trypsin solution consisted of 0.5 mg/mL trypsin in Tris saline (20 mM Tris-HCl, 50 mM NaCl, pH 7.5). The control solution was Tris saline or BSA (0.5 mg/mL) in Tris saline. After allowing reactions to proceed at RT for 3 h, they were terminated by washing the slides with deionized water. A fluorescence intensity ratio (FIR) was obtained from the ratio of fluorescence observed after trypsin digestion to that observed before digestion. The results demonstrate the feasibility of this system for high-throughput screening of the sequence selectivity of proteases, limited only by the numbers of synthetic peptides available.

The site-specific conjugation of fluorogenic substrates to glass surfaces should permit continuous kinetic analysis of protease activity and should be useful for screening potential protease inhibitors. These substrates can be used to determine substrate specificity, to provide valuable information about biological function, and also to help in the design of potent and selective substrates and inhibitors. A long excitation and emission wavelength are preferred in this microarray application since long wavelengths are less compromised by the auto-fluorescence of drug candidates, biological samples and some array substrates.

From the discussion above it will be appreciated that the invention can be embodied in various ways, including the following:

1. A surface modifying agent for the attachment of a biomolecule to a substrate, comprising a spacer having first and second ends; a substrate surface conjugating functional group attached to the first end of the spacer and configured to couple with a substrate surface; and a biomolecule conjugating functional group attached to the second end of the spacer and configured to couple to a biomolecule.

2. The agent of embodiment 1, wherein the substrate surface conjugating functional group attached to the first end of the spacer comprises an alkoxysilane functional group.

3. The agent of embodiment 2, wherein the alkoxysilane functional group comprises a trialkoxysilane.

4. The agent of embodiment 3, wherein the trialkoxysilane functional group comprises a triethyloxysilane functional group.

5. The agent of embodiment 1, wherein the substrate surface conjugating functional group attached to the first end of said spacer is selected from the group consisting essentially of a dihydrogen phosphate group, a thiol group and an alkyne group.

6. The agent of embodiment 1, wherein the biomolecule conjugating functional group attached to the second end of the spacer comprises an azide functional group.

7. The agent of embodiment 1, wherein the biomolecule conjugating functional group attached to the second end of the spacer is selected from the group consisting essentially of an alkene, an ester, a ketone, an aldehyde, a carbamate and a phosphane.

8. The agent of embodiment 1, wherein the spacer comprises a poly(ethylene glycol) molecule ranging in length from three ethylene glycol units to twenty ethylene glycol units.

9. The agent of embodiment 1, wherein the spacer comprises a molecule selected from the group of molecules consisting essentially of peptides, peptide mimics, nucleotides, nucleotide mimics and biopolymers.

10. A method of attaching a peptide to a substrate surface, comprising: (a) modifying a substrate surface with a modifying agent, said modifying agent comprising: a spacer having first and second ends; a substrate surface conjugating functional group attached to the first end of said spacer and configured to couple with a substrate surface; and a biomolecule conjugating functional group attached to said second end of said spacer and configured to couple to a biomolecule; and (b) reacting a biomolecule with the biomolecule conjugating functional group of the spacer; (c) wherein the spacer is coupled to the substrate at the first end and the biomolecule at the second end.

11. The method of embodiment 10, wherein the substrate surface conjugating functional group attached to the first end of the spacer is a silane selected from the group consisting essentially of an alkoxysilane group; a trialkoxysilane group and a triethyloxysilane group.

12. The method of embodiment 10, wherein the substrate surface conjugating functional group attached to the first end of the spacer is selected from the group consisting essentially of a dihydrogen phosphate group, a thiol group and an alkyne group.

13. The method of embodiment 10, wherein the biomolecule conjugating functional group attached to the second end of the spacer comprises an azide functional group.

14. The method of embodiment 10, wherein the biomolecule conjugating functional group attached to the second end of said spacer is selected from the group consisting essentially of an alkene, an ester, a ketone, an aldehyde, a carbamate and a phosphane.

15. The method of embodiment 10, wherein the spacer comprises a poly(ethylene glycol) molecule ranging in length from three ethylene glycol units to twenty ethylene glycol units.

16. The method of embodiment 10, wherein the spacer comprises a molecule selected from the group of molecules consisting essentially of peptides, peptide mimics, nucleotides, nucleotide mimics and biopolymers.

17. The method of embodiment 10, further comprising marking the biomolecule with a marker prior to reacting the biomolecule with the biomolecule conjugating functional group of the spacer.

18. The method of embodiment 17, wherein the marker comprises a fluorescent marker.

19. The method of embodiment 18, wherein the fluorescent marker comprises a 7-nitrobenz-2-oxa-1,3-diazol-4-yl fluorescent marker.

20. The method of embodiment 10, wherein the biomolecule is a molecule selected from the group of molecules consisting essentially of a peptide; a peptide analogue; a peptide mimic, a carbohydrate; a carbohydrate derivative; a lipid; a lipid derivative, a nucleic acid; a nucleic acid derivative and a nucleic acid protein complex.

21. The method of embodiment 10, wherein the substrate surface is selected from a group of substrate surfaces consisting of a glass surface, a silica surface; a silica gel surface; a metal surface; and a silicone surface.

22. The method of embodiment 10, wherein the substrate is selected from the group of substrates consisting essentially of polymer beads, agarose beads, and metallic oxide-based nanoparticles, glass cylinders, microarray elements, microfluidic components, and screening array components.

23. A method for producing a high density microarray, comprising: forming bifunctional linkers with a spacer having an alkoxysilane functional group attached to a first end of the spacer and an azide functional group attached to a second end of the spacer; reacting the alkoxysilane functional group of each spacer with a substrate surface; and coupling a biomolecule with an alkyne group with the azide functional group of each spacer.

24. The method of claim 23, wherein the alkoxysilane functional group comprises a trialkoxysilane.

25. The method of embodiment 23, wherein the trialkoxysilane functional group comprises a triethyloxysilane functional group.

26. The method of embodiment 23, further comprising marking the biomolecule with a marker.

27. The method of embodiment 26, wherein the marker comprises a fluorescent marker.

28. The method of embodiment 26, wherein the fluorescent marker comprises a 7-nitrobenz-2-oxa-1,3-diazol-4-yl fluorescent marker.

29. The method of embodiment 23, further comprising the step of hydrating a surface of the substrate before reacting the alkoxysilane functional group of each spacer.

30. The method of embodiment 23, wherein the spacer comprises a poly(ethylene glycol) molecule ranging in length from three ethylene glycol units to twelve ethylene glycol units.

31. The method of embodiment 23, wherein the biomolecule is a molecule selected from the group of molecules consisting essentially of a peptide; a carbohydrate; a carbohydrate derivative; a lipid; a lipid derivative, a nucleic acid; and a nucleic add protein complex.

32. The method as recited in embodiment 23, wherein the substrate surface is selected from a group of substrate surfaces consisting of a glass surface, a silica surface; a silica gel surface; a metal surface; and a silicone surface.

33. The method as recited in embodiment 23, wherein the substrate is selected from the group of substrates consisting essentially of polymer beads, agarose beads, and metallic oxide-based nanoparticles, glass cylinders, microarray elements, microfluidic components, and screening array components.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A surface modifying agent for the attachment of a biomolecule to a substrate, comprising:
a spacer having first and second ends;
a substrate surface conjugating functional group attached to the first end of said spacer and configured to couple with a substrate surface; and
a biomolecule conjugating functional group attached to said second end of said spacer and configured to couple to a biomolecule.

2. An agent as recited in claim 1, wherein the substrate surface conjugating functional group attached to the first end of said spacer comprises an alkoxysilane functional group.

3. An agent as recited in claim 2, wherein the alkoxysilane functional group comprises a trialkoxysilane.

4. An agent as recited in claim 3, wherein the trialkoxysilane functional group comprises a triethyloxysilane functional group.

5. An agent as recited in claim 1, wherein the substrate surface conjugating functional group attached to the first end of said spacer is selected from the group consisting essentially of a dihydrogen phosphate group, a thiol group and an alkyne group.

6. An agent as recited in claim 1, wherein the biomolecule conjugating functional group attached to the second end of the spacer comprises an azide functional group.

7. An agent as recited in claim 1, wherein the biomolecule conjugating functional group attached to the second end of said spacer is selected from the group consisting essentially of an alkene, an ester, a ketone, an aldehyde, a carbamate and a phosphane.

8. An agent as recited in claim 1, wherein the spacer comprises a poly(ethylene glycol) molecule ranging in length from three ethylene glycol units to twenty ethylene glycol units.

9. An agent as recited in claim 1, wherein the spacer comprises a molecule selected from the group of molecules consisting essentially of peptides, peptide mimics, nucleotides, nucleotide mimics and biopolymers.

10. A method of attaching a peptide to a substrate surface, comprising:
(a) modifying a substrate surface with a modifying agent, said modifying agent comprising:
a spacer having first and second ends;
a substrate surface conjugating functional group attached to the first end of said spacer and configured to couple with a substrate surface; and
a biomolecule conjugating functional group attached to said second end of said spacer and configured to couple to a biomolecule; and
(b) reacting a biomolecule with the biomolecule conjugating functional group of the spacer;
(c) wherein the spacer is coupled to the substrate at the first end and the biomolecule at the second end.

11. A method as recited in claim 10, wherein the substrate surface conjugating functional group attached to the first end of said spacer is a silane selected from the group consisting essentially of an alkoxysilane group; a trialkoxysilane group and a triethyloxysilane group.

12. A method as recited in claim 10, wherein the substrate surface conjugating functional group attached to the first end of the spacer is selected from the group consisting essentially of a dihydrogen phosphate group, a thiol group and an alkyne group.

13. A method as recited in claim 10, wherein the biomolecule conjugating functional group attached to the second end of the spacer comprises an azide functional group.

14. A method as recited in claim 10, wherein the biomolecule conjugating functional group attached to the second end of said spacer is selected from the group consisting essentially of an alkene, an ester, a ketone, an aldehyde, a carbamate and a phosphane.

15. A method as recited in claim 10, wherein the spacer comprises a poly(ethylene glycol) molecule ranging in length from three ethylene glycol units to twenty ethylene glycol units.

16. A method as recited in claim 10, wherein the spacer comprises a molecule selected from the group of molecules consisting essentially of peptides, peptide mimics, nucleotides, nucleotide mimics and biopolymers.

17. A method as recited in claim 10, further comprising:
marking the biomolecule with a marker prior to reacting the biomolecule with the biomolecule conjugating functional group of the spacer.

18. A method as recited in claim 17, wherein said marker comprises a fluorescent marker.

19. A method as recited in claim 18, wherein said fluorescent marker comprises a 7-nitrobenz-2-oxa-1,3-diazol-4-yl fluorescent marker.

20. A method as recited in claim 10, wherein the biomolecule is a molecule selected from the group of molecules consisting essentially of a peptide; a peptide analogue; a peptide mimic; a carbohydrate; a carbohydrate derivative; a lipid; a lipid derivative, a nucleic add; a nucleic acid derivative and a nucleic acid protein complex.

21. A method as recited in claim 10, wherein the substrate surface is selected from a group of substrate surfaces consisting essentially of a glass surface, a silica surface; a silica gel surface; a metal surface; and a silicone surface.

22. A method as recited in claim 10, wherein the substrate is selected from the group of substrates consisting essentially of polymer beads, agarose beads, and metallic oxide-based nanoparticles, glass cylinders, microarray elements, microfluidic components, and screening array components.

23. A method for producing a high density microarray, comprising:
forming bifunctional linkers with a spacer having an alkoxysilane functional group attached to a first end of the spacer and an azide functional group attached to a second end of the spacer;
reacting the alkoxysilane functional group of each spacer with a substrate surface; and
coupling a biomolecule with an alkyne group with the azide functional group of each spacer.

24. A method as recited in claim 23, wherein the alkoxysilane functional group comprises a trialkoxysilane.

25. A method as recited in claim 23, wherein the trialkoxysilane functional group comprises a triethyloxysilane functional group.

26. A method as recited in claim 23, further comprising:
marking the biomolecule with a marker.

27. A method as recited in claim 26, wherein said marker comprises a fluorescent marker.

28. A method as recited in claim 27, wherein said fluorescent marker comprises a 7-nitrobenz-2-oxa-1,3-diazol-4-yl fluorescent marker.

29. A method as recited in claim 23, further comprising the step of hydrating a surface of said substrate before reacting said alkoxysilane functional group of each linker.

30. A method as recited in claim 23, wherein the spacer comprises a poly(ethylene glycol) molecule ranging in length from three ethylene glycol units to twelve ethylene glycol units.

31. A method as recited in claim 23, wherein the biomolecule is a molecule selected from the group of molecules consisting essentially of a peptide; a carbohydrate; a carbohydrate derivative; a lipid; a lipid derivative, a nucleic add; and a nucleic acid protein complex.

32. A method as recited in claim 23, wherein the substrate surface is selected from a group of substrate surfaces consisting of a glass surface, a silica surface; a silica gel surface; a metal surface; and a silicone surface.

33. A method as recited in claim 23, wherein the substrate is selected from the group of substrates consisting essentially of polymer beads, agarose beads, and metallic oxide-based nanoparticles, glass cylinders, microarray elements, microfluidic components, and screening array components.
